(19) United States
(12) Patent Application Publication
(10) Pub. No.: US 2024/0341964 A1
(43) Pub. Date: Oct. 17, 2024

(54) TIBIAL COMPONENT OF A KNEE JOINT ENDOPROSTHESIS WITH TIBIAL PLATE AND ANCHORING STEM

(71) Applicant: WALDEMAR LINK GMBH & CO. KG, Hamburg (DE)

(72) Inventors: Eckhard BAUER, Kiel (DE); Helmut D. LINK, Hamburg (DE)

(73) Assignee: WALDEMAR LINK GMBH & CO. KG, Hamburg (DE)

(21) Appl. No.: 18/683,258

(22) PCT Filed: Aug. 5, 2022
(86) PCT No.: PCT/EP2022/072056

§ 371 Date: Feb. 13, 2024

(30) Foreign Application Priority Data
Aug. 16, 2021 (EP) 21000232.5

(51) Int. Cl. A61F 2/38; A61F 2/30; A61F 2/46

(52) U.S. Cl. CPC A61F 2/389; A61F 2/4637; A61F 2002/30329

(57) ABSTRACT

A tibial component of a knee joint endoprosthesis includes a laterally extending tibial plate, a distally projecting anchoring stem with a shaft piece, and at least one laterally extending wing-like extension. In an implantation state, the tibial plate and the anchoring stem are connected to one another by means of detachable fastening elements. The tibial plate is removable from the anchoring stem for an explantation state. At least one spacer that can be removed proximally is provided on the tibial plate, the end face of which forms a stop for the extension and is designed to form a stop between the extension and the tibial plate to create space with a defined minimum width as a saw slot space.

### FIELD AND BACKGROUND OF THE INVENTION

The invention relates to a tibial component of a knee joint endoprosthesis. The tibial component comprises a laterally extending tibial plate, a distally projecting anchoring stem with a shaft piece, and at least one laterally extending wing-like extension.

Knee joint prostheses usually have two components, one which is arranged at the proximal end of the tibia (tibial plateau) and another which is arranged at the distal end of the femur. Such prostheses are known and are available in different sizes and types so that they can be adapted to individual characteristics. Regarding the implantation, a distinction is made between cemented and non-cemented variants. Modular systems were introduced in order to be able to provide a suitable endoprosthesis for all variants (for example, U.S. Pat. No. 7,357,817 B2). They make it possible, for example, to select different tibial plates for an anchoring stem to be fastened in the tibia and, in particular, to choose between a cemented and a cementless version. The cementless version has the advantage that a high level of attachment can be achieved due to the ingrowing bone material, which extends the life of the prosthesis. The service life of such endoprostheses is limited, however, and revision surgeries in which the already implanted prosthesis, and in particular its tibial component, have to be removed are becoming increasingly frequent. The explantation often proves to be problematic in practice. A conservative explantation, in which as much bone material as possible is preserved and only a little has to be removed, is of considerable value for the patient. In the case of tibial components consisting of two parts, the problem of being able to remove the tibial plate conservatively even when a considerable ingrowth of bone material has already taken place arises. In addition, it often proves to be particularly difficult to remove the anchoring stem as well. Often this can lead to significant bone loss.

A known solution to this problem is to place the tibial plate on the anchoring stem by means of a central conical connection in such a way that a free space in the form of a saw slot space remains between them (EP 1 674 052 B1). A further plug-in connection is provided on the free end of the extension of the anchoring stem as a safeguard against any undesired twisting of the tibial plate relative to the anchoring stem. This additional plug-in connection is made from a material that can be cut, so that when the tibial plate is cut free by means of a saw inserted into the space for the saw slot, this additional plug-in connection can easily be cut. If the anchoring stem is not aligned exactly parallel to the tibial plate, which in practice can easily be happen in the event of asymmetrical stress, the free space narrows towards one of the outer ends due to the considerable lateral extension of the tibial plate. As a result, said free space is no longer sufficient for the saw blade, which makes it difficult to cut through the bone material.

### SUMMARY OF THE INVENTION

The object of the invention is to create an improved tibial component that avoids these disadvantages.

The solution according to the invention lies in the features of the independent claims. Advantageous developments are the subject-matter of the dependent claims.

In a tibial component of a knee joint endoprosthesis comprising a laterally extending tibial plate, a distally projecting anchoring stem with a shaft piece, and at least one laterally extending wing-like extension, wherein, in an implantation state, the tibial plate and the anchoring stem are connected to one another by means of detachable fastening elements, and wherein the tibial plate can be removed from the anchoring stem in the explantation state, at least one proximally removable spacer is provided on the tibial plate according to the invention, the end face of which forms a stop for the extension and that is designed to create a preferably slot-like free space with a defined minimum width between the extension and the tibial plate.

An anchoring stem of an endoprosthesis is understood to refer to a structure which is provided for anchoring the endoprosthesis in a bone, in particular a medullary cavity of the tibia, and which comprises a shaft and wing-like extensions arranged laterally thereon.

An implantation state is understood to refer to a mounted state of the tibial component that it has in the implanted state. Often this is also the state with which the implantation is carried out. This applies in particular in the case of a pre-assembly, for example when the anchoring stem and the tibial plate are already connected to one another.

An explantation state is understood to refer to a state that differs from the implantation state and is realized for the explantation.

The width of the slot-like free space is preferably dimensioned such that it is sufficient for the passage of a saw blade or a saw band in order to cut through the (bone) material located in the free space. The slot-like free space can thus form a saw slot space.

“Proximally removable” is understood to mean that the spacer can be removed from the free space from the proximal side without requiring access to the distal side. (The distal side is typically firmly attached to the bone by means of bone cement or ingrown bone material and is therefore difficult to access.)

Terms such as “distal” or “proximal” are anatomical terms for directional designations. They stand for a direction away from the center of the body and for a direction towards the center of the body. The term “lateral” refers to a sideways direction toward a left or right side of the body.

The invention is based on the finding that, thanks to the removable spacers, a positive control over the distances can also be achieved in the region of the respective end of the extension. The risk of a narrowing of the free space due to the often unavoidable misalignment as a result of asymmetric stress can thus be countered in a simple but effective way. The difficulty arises, however, that a sufficiently robust spacer is typically made of a material (such as titanium) that is difficult to cut with a bone saw. In this respect, there is a conflict of objectives. The invention solves this conflict of objectives by providing a spacer that is configured to be removable from the proximal side. This way, the spacer can be removed before the saw is used and any obstacles can be removed from slot-like free space being used as a saw slot. Nothing stands in the way of an unobstructed cutting process.

Since only bone material needs to be cut but no other materials (namely that of the spacer), no unwanted chips from exogenous material reach or get into the bone. This is another significant advantage, especially in terms of reducing the infection risk for the patient.

The fastening opening preferably has a width that is sufficient for the spacer to pass through, with the fastening opening preferably having a graduated design in which the width increases in the proximal direction. This simplifies the defined positioning and removal of the spacer, in particular if the graduated fastening opening has a shoulder that serves as a stop for the spacer.

In the implanted state, the spacer is expediently screwed into a fastening opening in the tibial plate. This way, the spacer can be implanted in a simple manner and easily removed, i.e. unscrewed, for explantation purposes. Furthermore, this offers the optional possibility of fine-tuning the distance, if desired, by screwing the spacer inward to different extents.

The spacer advantageously comprises an external thread which engages in an internal thread of the fastening opening. This way, the spacer can be fastened efficiently in the fastening opening. In addition, the external thread makes it possible to optimally use the installation space available.

The spacer is preferably designed in such a way that it has a dual function and also functions as a mount for a fastening screw which is to be actuated from the proximal end and which connects the anchoring stem to the tibial plate. This way, not only can a defined distance be set in a space-saving manner, but the tibial plate can also be fastened to the extension. The extension can thus fully develop its supporting and stiffening effect for the tibial plate.

The fastening screw is expediently arranged coaxially in the spacer, and preferably closes access to an actuating element of the spacer in the mounted state. On the one hand, the coaxial arrangement leads to a particularly space-saving design, which allows for larger fastening screws and thus a more robust connection in consideration of the installation space available. On the other hand, the coaxial arrangement offers the advantage that the screw head of the fastening screw can cover the upper side of the spacer almost completely. As a result, the upper side thus remains free from any impairments by foreign material, and, when the fastening screw is inserted, an actuation of the spacer is blocked, even if only unintentionally, due to lack of accessibility.

It has proven useful if a second extension is provided on the anchoring stem opposite the extension, which interacts on the tibial plate in a corresponding manner with a second spacer and a second fastening screw. A symmetrical arrangement of the extension is typically provided in order to achieve a symmetrical support of the tibial plate on both sides. However, a symmetrical arrangement is not mandatory; the length and/or angular position may differ as well. Preferably, the extensions are arranged so that they form an angle between them ranging from 100° to 170°. Thanks to the large angle, broad support is thus achieved which, however, since the angle deviates from 180°, is not in line with the shaft piece. Instead, the extensions preferably protrude in the posterior direction. Additional anterior or posterior support (anatomical terms for forward or backward direction as seen in the direction of the body) can thus also be provided, and as a result an even better support effect can be achieved by the anchoring stem.

It is expedient if the anchoring stem with the extensions has a planar upper side 85. This way, in conjunction with the typically planar distal underside of the tibial plate and the distance defined by the spacers, a uniform width of the saw slot space can be achieved over the entire reach of the extension.

An opening for a central screw with which the anchoring stem is fastened to the tibial plate is advantageously provided on the tibial plate. Using the central screw, a strong stress-transmitting connection can be provided between the tibial plate and the anchoring stem. Furthermore, the central screw makes it possible for the anchoring stem to be pulled towards the tibial plate against the effect of the spacers. This creates a certain tightening effect that offers additional protection, especially against any undesired loosening.

The distal side of the tibial plate is advantageously provided with a porous structure. This creates favorable conditions for the bone ingrowth and thus a good attachment of the tibial plate on the large contact surface with which the distal side rests flat on the bone. Preferably, the porous structure is designed to have interconnected pores deep within the material. The porosity thus goes beyond a superficial porosity and reaches deep into the material. Cavities are created in this way, which are connected to one another, and as a result of which particularly favorable conditions are created for the ingrowth of bone material. The pores are advantageously dimensioned in such a way that they have a width ranging from 0.1 to 1.5 mm, in particular between 0.7 and 1.3 mm. This results in a particularly favorable ingrowth behavior. It is expedient if the depth of the porous structure comprises at least one and up to three layers of pores, which typically means a preferred thickness of the porous structure of about 0.6 mm to 2.5 mm.

It is expediently provided that the porous structure covers the entire distal side of the tibial plate. This way, a solid ingrowth of bone material can be achieved across the entire distal surface of the tibial plate, resulting in an optimal connection of the tibial plate to the resected tibia head.

In view of the considerable stress on the knee joint, which is transmitted to the tibial plate via the condyles of the femur component, reinforcing ribs are expediently provided on the distal side of the tibial plate. A considerable reinforcement of the tibial plate, which is preferably designed to be thin, can thus be achieved. This can also counteract the effect that, as a result of the porous structure and the same outer thickness, the material thickness of the actually load-bearing, solid part of the tibial plate, and thus also its load-bearing capacity, is reduced.

Preferably, the reinforcing ribs on the distal side of the tibial plate extend radially from a central region to the edge of the tibial plate. This effectively provides reinforcement along the main stress lines of the tibial plate. The reinforcing ribs can be made so strong that they jut out and in particular protrude from the porous structure. However, it can also be provided that the reinforcing ribs are countersunk into the porous structure. This has the advantage that there is a continuous porous structure on the surface, which promotes an extensive ingrowth of bone material.

A circumferential edge is advantageously provided for the porous structure on the distal side of the tibial plate. The edge provides for a clear delimitation of the porous structure. However, a further advantage of the edge lies in the fact that it is possible to prevent the mechanically more sensitive porous material from breaking out of the porous structure, particularly in the outer regions of the porous structure.

Expediently, the width of the edge is only dimensioned in such a way that the porous structure occupies at least half, preferably more than 60%, of the surface of the distal side of the tibial plate. This avoids that too large a part of the distal underside of the tibial plate is not available for the porous structure due to an overly wide edge. Otherwise, if the tibial plate is small in particular, this can otherwise become a problem with regard to a sufficiently secure fastening of the tibial plate due to ingrowing bone material. The selected lower limit ensures that there is sufficient space for the ingrowth of bone material.

The invention further includes a modular tibial component system of knee joint endoprostheses, comprising one or more of the tibial components described above, wherein at least two different tibial plates are provided, which can be optionally connected to an anchoring stem, the tibial plates differing in terms of their design, in particular with regard to their bone-contacting surfaces. Various components can be combined with such a modular system, for example different tibial plates with an anchoring stem, and/or a suitable anchoring stem is selected from a selection of a plurality of anchoring stems and combined with one of the tibial plate. Preferably, the tibial component system comprises different tibial plates in different sizes and/or at least two different anchoring stems in different sizes. Overall, with such combinations, the individual requirements of each patient can be taken into consideration to a large extent with a manageable number of different components.

With a view to providing a solid fastening across all sizes, it is expedient that, for tibial plates of different sizes, the width of the edge is narrower for smaller sizes. Due to the narrower design of the edge, more surface area is provided for the smaller sizes, which can be used for a porous structure. This way, a sufficiently secure attachment of the distal side of the tibial plate to the bone head is ensured by ingrowing bone material, even in the case of the smaller sizes. In this context, sizes refers to relative qualitative indications in relation to the dimensions of the individual components, for example “S” for a small size, “M” for a medium size, “X” for a large and “XL” for an extra-large model. It goes without saying that more than four different sizes can be provided as well.

The invention also relates to an instrument set for the tibial component, which instrument set comprises a saw with a saw blade or saw band as extraction tools, with the saw width being at the most as large as the minimum width of the free space, and a cutting sleeve, the inner contour of which is designed to accommodate the lateral outer contour of the anchoring stem with its extensions, and a connection element which can be connected to the shaft part of the anchoring stem with a high tensile strength. The “lateral outer contour” is understood to refer to the maximum outer contour in a plane perpendicular to the proximal-distal direction. This describes a laterally circumferential wrap-around.

Thanks to the extraction tools, a patient-friendly explantation can be achieved. The saw with a saw blade or saw band of a specified width ensures that it can penetrate the saw slot and cause the desired cutting through of bone material on the distal side of the tibial plate so that the tibial plate can be cut free and removed. In a subsequent step, the immediate vicinity of the anchoring stem can be separated from the proximal end of the actual bone material of the tibia head or the tibia as a whole by means of the cutting sleeve. By inserting the cutting sleeve, the anchoring stem, particularly its protruding proximal region, can be cut free from the surrounding bone material. The connection element, which can be connected to the shaft part of the anchoring stem with a high tensile strength, offers the advantage that it provides a starting point in order to be able to pull out the part of the anchoring stem that is still in the tibia, in particular after it was detached from its surroundings. The set of instruments thus facilitates the removal of the anchoring stem, while at the same time protecting the end of the bone by detaching the anchoring stem free in a defined manner thanks to the cutting sleeve according to the invention.

The invention also extends to a method for extracting the tibial component, wherein, after the tibial plate has been exposed, it is provided that the fastening screws are loosened and removed from the tibial plate, the spacers are removed from the tibial plate using a saw with a saw blade or saw band the saw width of which is less than or equal to the minimum width of the free space, ingrown bone material is cut along the distal side of the tibial plate, and the tibial plate is removed. This particularly advantageously includes the steps of placing a cutting sleeve, the inner contour of which is designed to accommodate the lateral outer contour of the anchoring stem with its extensions, on a resection plane that is exposed by removing the tibial plate, and cutting the anchoring stem from free by moving the cutting sleeve distally, preferably by means of a tensioning device, which is arranged on a connection element fastened proximally to the shaft piece.

The method also optionally includes connecting an extraction instrument to the shaft piece and/or the connection element, extracting the anchoring stem that has been detached from its surroundings by applying a tensile force, preferably by means of a sliding hammer.

Sufficient actuating force can be gently applied with the sliding hammer to pull the anchoring stem that has been cut free from the tibia head. This way, the tibial component can be explanted with relatively little bone loss.

For a more detailed explanation, reference is made to the above description of the tibial component and the steps involved in the explantation.

### BRIEF DESCRIPTION OF THE DRAWINGS

The invention is explained in more detail below with reference to the drawing which depicts an advantageous illustrative embodiment, and in which:

FIG. 1 is a front view of an embodiment of the tibial component of a knee joint endoprosthesis;

FIG. 2A, B are exploded views of FIG. 1 with separated or connected tibial plate and anchoring stem;

FIG. 3 is an exploded perspective view of FIG. 2A viewed from the oblique distal direction;

FIG. 4 is a lateral view of FIG. 1;

FIG. 5 is a perspective view showing partially assembled spacers and fastening screws;

FIG. 6 is a perspective view showing the tibial component and an oscillating saw at the free space;

FIG. 7 is a perspective view with the tibial component and a band saw at the free space;

FIG. 8A-C are perspective distal oblique views of the tibial plate with a porous structure in three variants;

FIG. 9 is a plan view distal to FIG. 8A;

FIG. 10 is an enlarged sectional view of FIG. 9;

FIG. 11 illustrates tibial components of a modular tibial component in different sizes; and

FIG. 12A-I illustrate instruments and procedural steps for an explantation.

### DETAILED DESCRIPTION OF THE INVENTION

The invention is explained below using an example of a tibial component of a knee joint endoprosthesis. It is designed to be implanted at the proximal bone end (bone head) of a tibia. The tibial component designated in its entirety by the reference numeral 9 preferably consists of a titanium alloy. However, it can also consist of another biocompatible material, for example cobalt-chromium-molybdenum (CoCrMo).

The knee joint endoprosthesis comprises a femoral component (not shown) to be arranged on the femur bone (not shown) and the tibial component 9 to be arranged on the proximal end of a tibia bone 99. It includes a tibial plate 91, which is arranged in a laterally extended manner on a resected tibia bone head. A receiver is provided on the proximal side (top side) of the tibial plate 91 in which a bearing piece (not shown) of the knee joint endoprosthesis is to be arranged. The opposite distal side 92 of the tibial plate 91 is shaped to abut the surface of the tibia bone head 99 and is therefore provided with a porous structure for the ingrowth of bone material.

A distally protruding anchoring stem 8 is provided to fasten the tibial plate 91. It comprises a distally adjoining shaft piece 82 and a conical piece 81. The conical piece 81 is designed to accommodate a plug-in shaft that protrudes into the medullary canal of the tibia. Laterally, the shaft piece 82 is adjoined by a wing-like extension 83 both on the left and on the right side, at the free end of which a clamping sleeve 84 is arranged. The wing-like extensions 83 act as support arms for the tibial plate 91. The tibial plate 91 is thus connected to the anchoring stem 8 at three points, namely centrally on the shaft piece 82 and on the left and right with the clamping sleeves 84 arranged on the free ends of the wing-like extensions 83. For this purpose, two screw holes are provided on the tibial plate 91 on the left and right as fastening openings 94, which are aligned with the respective clamping sleeve 84.

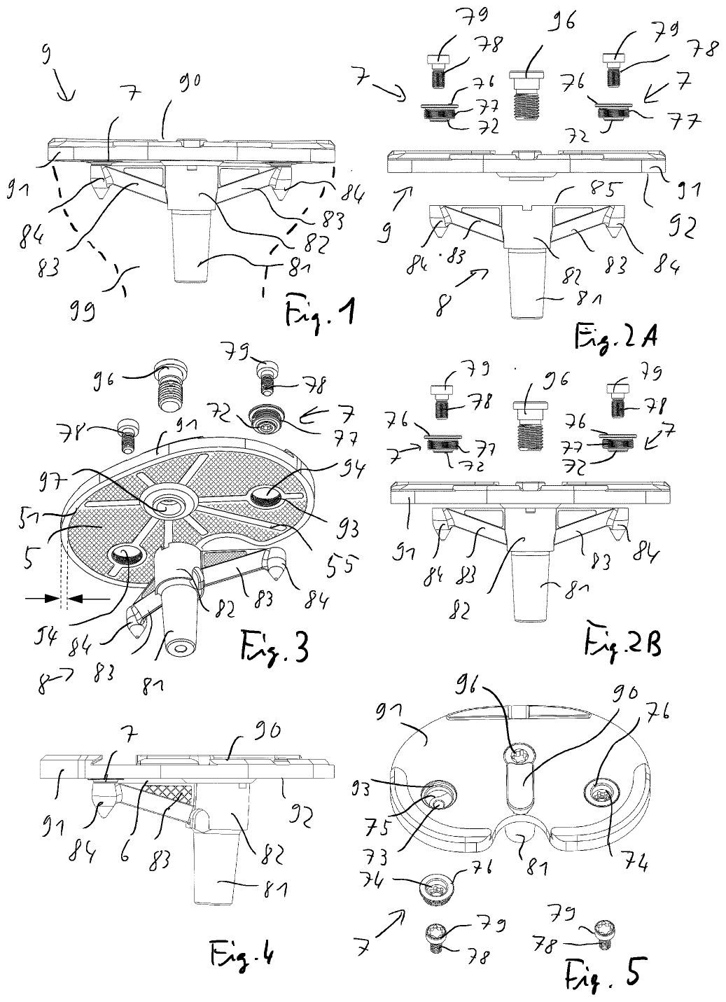

The wing-like extensions 83 are not flush with the underside 92 of the tibial plate 91, but are held at a defined distance. Spacers 7 with an external thread 77 are screwed into the fastening openings 94 (see FIGS. 1 and 4), which create a defined distance from the clamping sleeves 84.

This is explained in more detail below with reference to FIG. 2A, B and FIG. 3. Three openings are provided in the tibial plate 91 which are used to connect the tibial plate 91 to the anchoring stem 8 and its wing-like extension 83. A central opening 97 is provided in the middle, which is designed to accommodate a relatively large central screw 96. This screw serves to create a connection to the anchoring stem 8 via which the main stress acting on the tibial plate 91 via the knee joint of the knee joint endoprosthesis is transferred into the anchoring stem 8. The central opening 97 may be flanged at its mouth for reinforcement purposes and to provide a bearing surface. The fastening openings 94 are also offset from the lateral edges of the tibial plate 91. They have an internal thread 93 and are positioned in such a way that they are aligned with the clamping sleeves 84 at the end of the wing-like extensions 83. The fastening openings 94 are preferably each provided as a stepped bore, the proximal region of which has a greater width.

The spacers 7 with their external threads 77 are designed to be screwed into the internal threads 93 of the fastening openings 94. When the tibial component 9 is in the implanted state, they are screwed in far enough that their distal end face 72 protrudes from the distal underside 92 of the tibial plate 91. Thus, in the implanted state, the end faces 72 of the spacers 7 rest on the proximal side of the clamping sleeves 84. This way, a measure for the distance is set by the protruding end face 72 of the spacers 7. To ensure that the distance does not have to be set precisely by hand every time, a laterally protruding circumferential edge 76 is expediently formed on the spacers 7 in the proximal region. The thickness of this circumferential edge 76 and the length of the spacer 7 are matched to one another in such a way that the end face 72 protrudes from the distal underside 92 of the tibial plate 91 by the desired distance when the circumferential edge 76 comes to rest on the tibial plate 91. This placement can be made on the proximal top of the tibial plate 91. However, it is preferred if the spacer 7 is sunk into the respective fastening opening 94 and the circumferential edge 76 rests on a shoulder 75 of the stepped bore. This way, the surgeon does not have to worry about setting the distance, but rather screws the spacer 7 inward until the circumferential edge 76 rests on the shoulder 75 in the respective fastening opening 94. This automatically determines the correct protrusion of the end face 72.

If the tibial plate 91 is then mounted on the anchoring stem 8 in a manner known per se and the central screw 96 is tightened, the clamping sleeves 84 come to rest on the protruding end face 72 of the spacers 7, which then necessarily creates the defined distance and the desired free space 6 with a defined distance up to the outer free ends of the wing-like extension 83.

Fastening screws 78 for the spacers 7 are additionally provided for a better connection. For this purpose, the spacer 7 is designed as a sleeve with a coaxial inner opening 73 through which the fastening screw 78 is inserted. The fastening screw 78 can thus engage with its thread in the corresponding mating thread of the clamping sleeve 84, and, by tightening the fastening screw 78, the clamping sleeve 84 is clamped against the spacer 7 at the defined distance. The anchoring stem 8 is thus additionally fastened to the tibial plate 91 and secured.

This way, a defined distance is set between the underside 92 of the tibial plate 91 and the wing-like extensions 83 of the anchoring stem 8. With the defined distance, the slot-like free space 6 is created between the tibial plate 91 and the anchoring stem 8 with its wing-like extensions 83, which acts as a saw slot. This is shown in FIGS. 1 and 4, and the free space 6 acting as a saw slot is highlighted by the ellipses shown in dashed lines.

The tibial component 9 is in the implanted state with the pre-assembly of the anchoring stem 8 to the tibial plate 91 that has taken place in this way.

The spacers 7 must be removed for the explantation state. To this purpose, the fastening screws 78 are first loosened and removed. As a result, a screwdriver receiver 74 covered by the head 79 of the fastening screw 78 is accessible on the upper side of the spacers 7. This makes it possible to remove the spacers 7 from the fastening openings 94 in the next step. This state is shown in FIG. 6. It can be seen that the free space 6 between the top 85 of the wing-like extension 83 of the anchoring stem 8 and the bottom 93 of the tibial plate 91 is now laterally accessible for a saw blade 31. Bone material that has grown into the free space 6 can thus be cut with the saw blade 31 of an oscillating bone saw, and the tibial plate 91 can thus be cut free.

An alternative to this is shown in FIG. 7. A band saw 32 is used instead of a saw with a saw blade 31. It is provided with a pulling instrument 33, 33′ at each of its two ends. By moving the pulling instruments 33, 33′ back and forth, the saw blade 32 can cut through the bone material that has grown in the free space 6 and thus cut the tibial plate 91 free.

The tibial plate 91 is provided with a porous structure 5 on its distal side 92 (see FIG. 9). For this purpose, one (or more) large, pocket-like recesses 95 are formed on the underside, in which the porous structure 5 is arranged. The porous structure 5 is designed to have interconnected pores deep within the material (the porous structure). Cavities are created in this way, which are connected to one another, resulting in favorable conditions for the ingrowth of bone material. For this purpose, the pores are preferably range in size from 0.4 to 1 mm. The depth of the recess 95 is about 1 to 2.5 mm (see FIG. 10). The porous structure 5 can thus comprise at least one and up to three layers of pores which are connected to one another at a given depth.

The large pocket-like recess 95 is surrounded by a circumferential edge 51 (see FIG. 3). The edge has a width of about 1.0 to 2.5 mm. The distal side 92 of the tibial plate 91 is provided with the porous structure 5 almost over its entire surface (apart from the fastening holes 94, 97), as shown in particular in FIG. 8A. Variants are shown in FIGS. 8B and 8C in which reinforcing ribs 55 are provided, which extend radially from the central opening 95 to the edge 51. In the variant shown in FIG. 8C, however, the reinforcing ribs do not reach the edge 51, but instead, porous connecting surfaces 54 for the porous structure 5 are provided on the edge 51 on each side of the respective reinforcing ribs 55. In the variant shown in FIG. 8B, the reinforcing ribs 55′ are sunk into the porous structure, resulting in a continuous porous structure, at least in the region near the surface.

Tibial plates 91 of different sizes of a preferably modular tibial component system are shown in FIG. 11. Six different sizes starting from the smallest size (9-1) are shown. Two main reinforcing ribs 55 are provided for the smallest size. Beginning with size 9-2, first additional ribs 56 are provided for the medium sizes 9-3, 9-4, and 9-5 to provide further stiffening. The largest size (9-6) comprises second additional ribs 57 due to the higher stress to be expected. The main reinforcing ribs 55 each enclose the fastening openings 94 in order to compensate for the weakening of the tibial plate 91 resulting from the opening there.

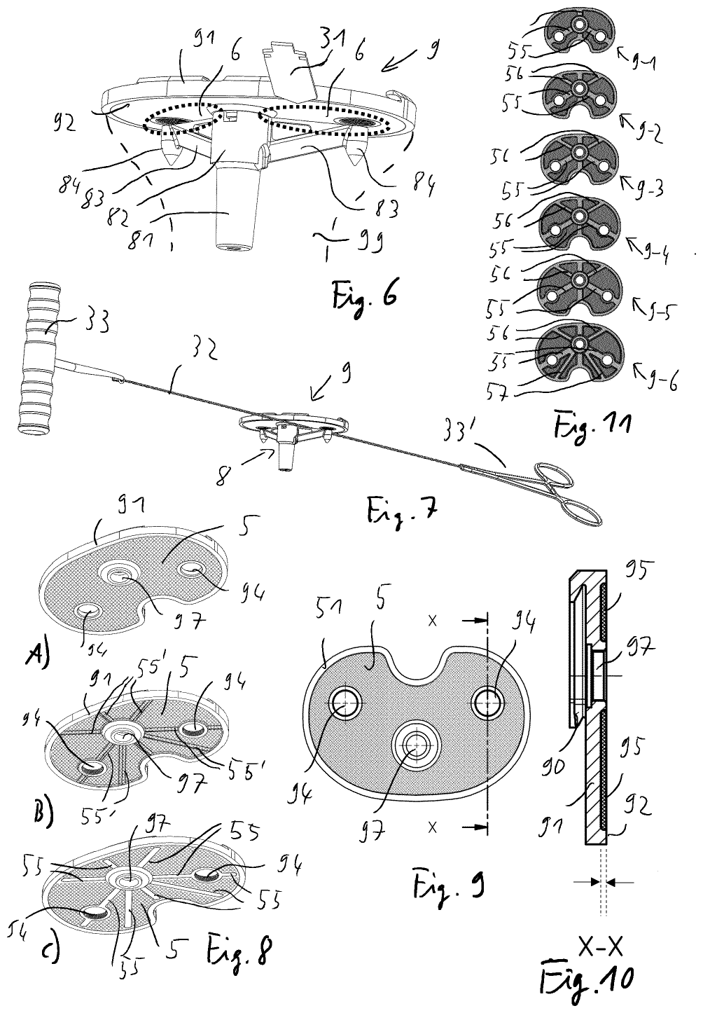

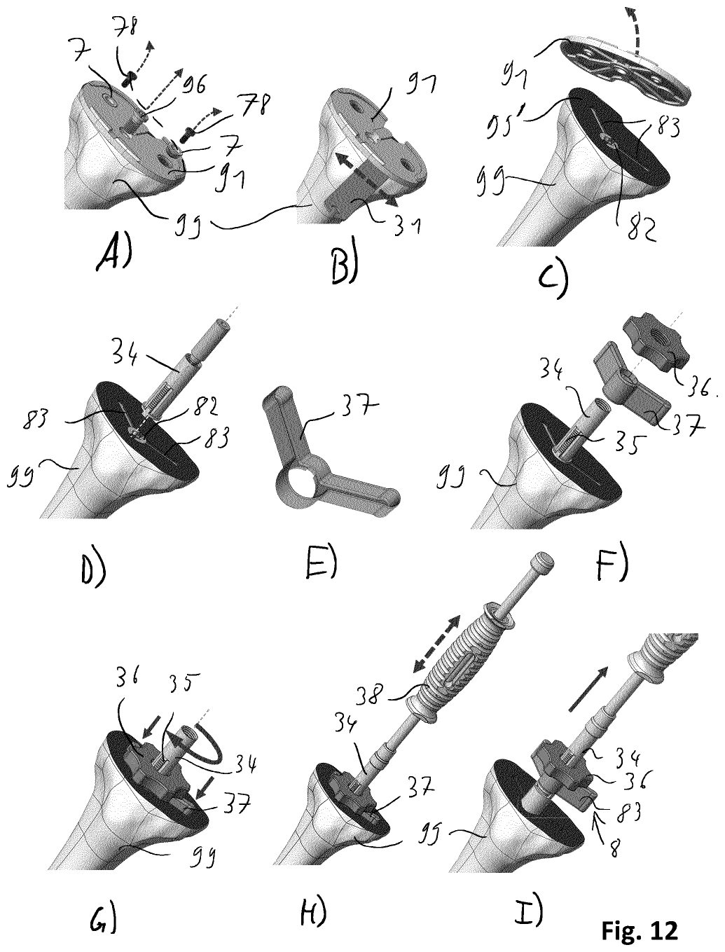

Reference is now made to FIG. 12, where method steps for the explantation of the tibial component 9 are shown. In a first step, the fastening screws 78 and the central screw 96 are to be unscrewed and removed. This is illustrated in FIG. 12A. In the next step, the spacers 7 are removed, an intermediate state being shown in FIG. 5, in which the spacer 7 has been removed and the other spacer 7 is still in the tibial plate 91. The completion of this step with the spacers 7 removed is shown in FIG. 12B. The free space 6 between the lower, distal side 92 and the upper edge 85 of the anchoring stem 8 with its extensions 83 is now completely open, as shown in FIG. 6 (refer to the areas marked with a dotted ellipse, which show the free space 6 without the spacer 7 having been removed in the meantime). This cannot be seen in FIG. 12B, since the free space 6 is located within the tibia 99. To detach the tibial plate from its surroundings, the saw blade 31 of a saw is moved into the free space 6, which now forms a saw slot. By moving the saw back and forth, the tibial plate 91 is cut free and can be removed in the next step, as shown in FIG. 12C.

In order to also remove the anchoring stem 8, a rod-like connection element 34 is screwed onto the shaft piece 82 and firmly connected in the next step, as shown in FIG. 12D. In the next step, a cutting sleeve 37 (see FIG. 12E), which comprises as the wrap-around the outer contour of the anchoring stem 8 with its lateral extensions 83, is placed on the connection element 34. Further, a tensioning device formed by a male thread 35 on the connection element 34 and a hand wheel 36 with a female thread is attached as shown in FIG. 12F. Turning the hand wheel 36 operates the tensioning device and drives the cutting sleeve 37 into the tibia head as shown in FIG. 12G. In the process, the cutting sleeve 37 cuts through the bone tissue laterally holding the anchoring stem 8 with its extensions 83. In the next step, a sliding hammer 38 as an extraction element is connected to the connection element 34 as shown in FIG. 12H. By operating the sliding hammer 38, the anchoring stem 8, which has been cut free and loosened by the cutting sleeve 37, can be driven out proximally and finally pulled out, as shown in FIG. 121.

1. A tibial component of a knee joint endoprosthesis, comprising a laterally extending tibial plate, a distally projecting anchoring stem with a shaft piece, and at least one laterally extending wing-like extension,
wherein, in an implantation state, the tibial plate and the anchoring stem are connected to one another by means of detachable fastening elements, and wherein the tibial plate can be removed from the anchoring stem in an explantation state,
wherein
at least one spacer that can be removed proximally is provided on the tibial plate, an end face of which spacer forms a stop for the extension and is designed to create a preferably slot-like free space with a defined minimum width.

2. The tibial component according to claim 1, wherein the fastening opening has a width sufficient for passage of the spacer, wherein the spacer is screwed into a fastening opening of the tibial plate in the implanted state, and wherein the fastening opening is graduated with a width increasing in the proximal direction.

3. The tibial component according to claim 2, wherein the spacer has an external thread which engages in an internal thread of the fastening opening.

4. The tibial component according to claim 2, wherein the spacer has a dual function and also functions as a mount for a fastening screw, which is to be actuated from the proximal end and which connects the anchoring stem to the tibial plate.

5. The tibial component according to claim 4, wherein the fastening screw is arranged coaxially in the spacer, wherein the fastening screw closes access to an actuating element of the spacer in a mounted state.

6. The tibial component according to claim 1, wherein a second extension is provided on the anchoring stem opposite the extension, which interacts on the tibial plate in a corresponding manner with a second spacer and a second fastening screw, wherein the extensions preferably form an angle between 100° and 170°.

7. The tibial component according to claim 1, wherein the anchoring stem has a planar upper side.

8. The tibial component according to claim 1, wherein an opening for a central screw is provided on the tibial plate with which the anchoring stem is fastened to the tibial plate.

9. The tibial component according to claim 1, wherein the tibial plate is provided on its distal side with a porous structure, which preferably comprises interconnected pores in a depth of the tibial plate material, which more preferably has a width from 0.1 to 1.5 mm.

10. The tibial component according to claim 9, wherein the porous structure fully covers the distal side of the tibial plate.

11. The tibial component according to claim 9, wherein protruding reinforcing ribs are provided on the distal side of the tibial plate and extend radially from a central region to an edge of the tibial plate.

12. The tibial component according to claim 9, wherein reinforcing ribs sunk into the porous structure are provided on the distal side of the tibial plate and extend radially from a central region to the edge of the tibial plate.

13. The tibial component according to claim 9, wherein a circumferential edge for the porous structure is provided on the distal side of the tibial plate.

14. The tibial component according to claim 13, wherein the width of the edge is only dimensioned in such a way that the porous structure covers at least half, preferably more than 60%, of a surface of the distal side of the tibial plate.

15. A modular tibial component system of knee joint endoprostheses, comprising one or more tibial components according to claim 1, wherein it has at least two different tibial plates which can be optionally connected to an anchoring stem, the tibial plates differing in terms of their design, particularly with regard to their bone-contacting surfaces.

16. The modular tibial component system according to claim 15, wherein the system comprises various tibial plates in different sizes and/or comprises at least two different anchoring stems in different sizes.

17. The modular tibial component system according to claim 16, wherein, having differently sized tibial plates with a porous structure, the width of an edge around the porous structure decreases at its distal side.

18. An instrument set for a tibial component according claim 1, wherein the instrument set comprises, as extraction tools,
a saw with a saw blade or a saw band, with a sawing width being at the most as large as the defined minimum width of the free space, and comprising:
a cutting sleeve, an inner contour of which is designed to accommodate the lateral outer contour of the anchoring stem with its extensions; and
a connection element, which can be connected to the shaft piece of the anchoring stem with a high tensile strength.

19. A method for extracting a tibial component according claim 1, wherein, after the tibial plate has been exposed:
loosening the fastening screws;
removing the fastening screws from the tibial plate;
removing the spacers from the tibial plate;
cutting, by means of a saw with a saw blade or saw band, bone material that has grown in the free space along a distal side of the tibial plate, a saw width of which is at most as large as the defined minimum width of the free space; and
removing the tibial plate.

20. The method according to claim 19, wherein a cutting sleeve, an inner contour of which is designed to accommodate the lateral outer contour of the anchoring stem with its extensions, is placed on a resection plane exposed by removing the tibial plate, and the anchoring stem is cut free by moving the cutting sleeve distally, by means of a clamping device, which is arranged on a connection element fastened proximally to the shaft piece.

21. The method according to claim 20, wherein an extraction instrument is connected to the shaft piece or the connection element, and the extraction of the anchoring stem, which has been cut free by applying a tensile force, by means of a sliding hammer.
